(19) United States
(12) Patent Application Publication
(10) Pub. No.: US 2017/0000908 A1
(43) Pub. Date: Jan. 5, 2017

(54) A MICELLAR PARTICLE

(71) Applicants: AGENCY FOR SCIENCE, TECHNOLOGY AND RESEARCH, Singapore (SG); NATIONAL UNIVERSITY OF SINGAPORE, Singapore (SG)

(72) Inventors: Xu LI, Singapore (SG); Changliang REN, Singapore (SG); Yu ZHANG, Singapore (SG); John WANG, Singapore (SG)

(21) Appl. No.: 15/100,270

(22) PCT Filed: Nov. 27, 2014
(86) PCT No.: PCT/SG2014/000562

§ 371 Date: May 27, 2016

(30) Foreign Application Priority Data
Nov. 27, 2013 (SG) 201308798-6

(51) Int. Cl. A61K 49/00; A61K 9/51

(52) U.S. Cl. CPC A61K 49/0093; A61K 9/5115; A61K 49/0039; A61K 49/0021; B82Y 30/00

(57) ABSTRACT

There is provided a micellar particle having at least a core-shell configuration, wherein a fluorescent molecule is encapsulated within said core and wherein said shell is formed from an inorganic compound.

### TECHNICAL FIELD

The present invention generally relates to a micellar particle. The present invention also relates to a process for forming a particle.

### BACKGROUND

Fluorescent probes are commonly used in real-time biological imaging to observe many sub-cellular structures or intracellular events such as protein-receptor interaction. Among all of the probes available, organic dye molecules are most widely used due to their small size and ease of use. However, most of the organic dyes suffer from either low solubility in, water or poor photochemical stability such as rapid photobleaching under repeated exposure, which limit their use for long-term biological studies. Extensive studies have been made to enhance the water solubility and photostability of such dye molecules by modifying the dye molecules with bulky dendrimers. However, the preparation of those water-soluble dye molecules required multiple-step laborious synthetic work with low yield, which largely limits their widespread application.

Other fluorescent probes used include quantum dots, which demonstrate high quantum yield, photostability and wide absorption spectrum with narrow emission line. However, quantum dots cannot be used in biomedical applications due to the use of toxic heavy metals such as cadmium and selenide. Thus, the applicability of quantum dots in bioimaging is heavily limited.

An alternate fluorescent probe that does not contain any toxic components is a nanocapsule which is basically a core-shell particle that encapsulates different dye molecules in the core. Due to the presence of the different dye molecules, the nanocapsule is capable of possessing different and distinctive light-emitting properties. Compared with other reported capsules such as liposomes or micelles which rapidly release the contained entities when introduced into a host and lead to instability of the capsules in vivo, the nanocapsules provide a much lower dye and oxygen permeability due to the more condensed structure on the shell, resulting in a higher photostability for most dyes. However, such nanocapsules are susceptible to clearance by the reticuloendothelial system, which limits the circulation duration of the nanocapsules in vivo.

There is a need to provide a fluorescent probe that overcomes, or at least ameliorates, one or more of the disadvantages described above.

### SUMMARY

According to a first aspect, there is provided a micellar particle having at least a core-shell configuration, wherein a fluorescent molecule is encapsulated within the core and wherein the shell is formed from an inorganic compound.

The micellar particle may include a corona portion that extends outwards from the shell, hence the micellar particle may have a core-shell-corona configuration. The micellar particle may then be termed as a core-shell-corona micellar particle.

Advantageously, the disclosed particle may be colloidally stable. The disclosed particle may have a diameter lesser than 100 nm. Hence, the disclosed particle may evade clearance by the reticuloendothelial system, leading to prolonged circulation time in vivo. The disclosed particle may be photoluminescent without using any toxic heavy metals. Hence, the disclosed particle may have low or no cytotoxicity to a biological host. The disclosed particle may have a high quantum yield. The disclosed particle may be less vulnerable to photobleaching leading to good photostability. The disclosed particle may be easily modified with external surface modification with desired ligand(s).

### DEFINITIONS

The following words and terms used herein shall have the meaning indicated:

The terms “micelle” or “micellar particle” as used herein refer to an assembly of amphiphilic block copolymers into a microphase separated, core/shell/corona or core/corona architecture in a selective organic solvent. A micelle is an aggregate of amphiphilic polymers dispersed in a liquid. A typical micelle in an aqueous solution forms an aggregate with the hydrophilic components in contact with the surrounding solvent (forming the corona portion of the micelle), sequestering the hydrophobic components in the micelle centre (forming the core portion of the micelle). Micelles are approximately spherical in shape, but other shapes such as ellipsoids, cylinders, and rods are also contemplated. The shape and size of a micelle is a function of the molecular geometry of its molecules and solution conditions such as concentration, temperature, pH, and ionic strength. The disclosed micellar particle may comprise a shell portion that is at least partially formed at the interface between the core portion and corona portion of the micellar particle. Hence, the disclosed micellar particle may have a core-shell-corona configuration. The shell portion may be comprised of an inorganic component or compound.

The term “polymer” or “polymeric” as used herein refers to a molecule having two or more monomeric repeat units. It includes linear and branched polymer structures, and also encompasses cross-linked polymers as well as copolymers (which may or may not be cross-linked), thus including block copolymers, alternating copolymers, random copolymers, and the like. Hence, the term “amphiphilic polymer” refers to a polymer which comprises at least a hydrophilic part (or block) and at least a hydrophobic part (or block).

The term “silica” as used herein refers to oxide of silicon having the approximate chemical formula SiO₂, without regard to shape, morphology, porosity, and water or hydroxyl content.

The term “fluorescent molecule” as used herein refers to a molecule with non-repetitive structural units that is capable of emitting a fluorescence when excited with light or other electromagnetic radiation.

The term “nano” as used herein, when referring to a dimension or a parameter, refers to the size of that dimension or parameter being in the nano-range, or less than about 500 nm, less than about 200 nm or less than about 100 nm.

The term “solvent” as used herein refers to a liquid which is used to partially or completely dissolve or disperse a compound at a given concentration such that a solution or dispersion is formed respectively.

The term “nanocapsule” as used herein refers to a particle having at least a core-shell configuration in which the core is hollow and which is loaded with a cargo. The cargo may be present as substantially discrete particles within the core and may not interact with the core or shell of the particle. Hence, the cargo may not substantially entangle with, or be embedded in or absorbed onto the core or the shell of the nanocapsule.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 0.1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF EMBODIMENTS

Exemplary, non-limiting embodiments of a micellar particle will now be disclosed. The micellar particle may have at least a core-shell configuration, wherein a fluorescent molecule is encapsulated within the core and wherein the shell is formed from an inorganic compound.

The micellar particle may comprise a corona that extends outwards from the shell, hence the micellar particle may have a core-shell-corona configuration. The micellar particle may then be termed as a core-shell-corona micellar particle.

The micellar particle may comprise an amphiphilic polymer that forms a micelle with a core-corona architecture. The shell may be formed of an inorganic compound in which the shell at least partially encapsulates the core portion of the micelle.

The amphiphilic polymer may have hydrophilic and hydrophobic components. The amphiphilic polymer may be a block copolymer or a block terpolymer. The amphiphilic polymer may be a copolymer having at least two blocks or a terpolymer having at least three blocks. The amphiphilic polymer may be a copolymer having two blocks (forming a diblock copolymer) or a copolymer having three blocks (forming a triblock copolymer). The diblock copolymer may have a hydrophilic block and a hydrophobic block. The triblock copolymer may have alternating blocks of hydrophilic block, hydrophobic block and hydrophilic block (where the hydrophilic blocks may be comprised of the same polymer or different polymers) or alternating blocks of hydrophobic block, hydrophilic block and hydrophobic block (where the hydrophobic blocks may be comprised of the same polymer or different polymers). The amphiphilic polymer may be biocompatible.

The hydrophobic component of the amphiphilic polymer may form the core of the micellar particle and the hydrophilic component of the amphiphilic polymer may form the corona of the micellar particle.

The copolymer may have a backbone formed from hydrophobic segments, with hydrophilic segments attached to the hydrophobic backbone at various points. The copolymer may have a backbone formed from hydrophilic segments, with hydrophobic segments attached to the hydrophilic backbone. The backbone may also be formed from both hydrophobic and hydrophilic segments, with one or both of hydrophilic and hydrophobic graft segments attached thereto.

The amphiphilic polymer may comprise a polyethylene-oxide monomer. The amphiphilic polymer may be selected from polyethylene-oxide (PEO) functionalized polymers, such as polyhedral oligosilsesquioxanes-PEO (POSS-PEO), PEO-polyhydroxybutyrate-PEO (PEO-PHB-PEO), polylactic-acid-PEO (PLA-PEO), PEO-PLA-PEO, PEO-polypropyleneoxide-PEO (PEO-PPO-PEO), polydimethylsiloxane-graft-PEO (PDMS-graft-PEO) or polystyrene-PEO (PS-PEO). The chemical structures of the various exemplary amphiphilic polymers are shown below:

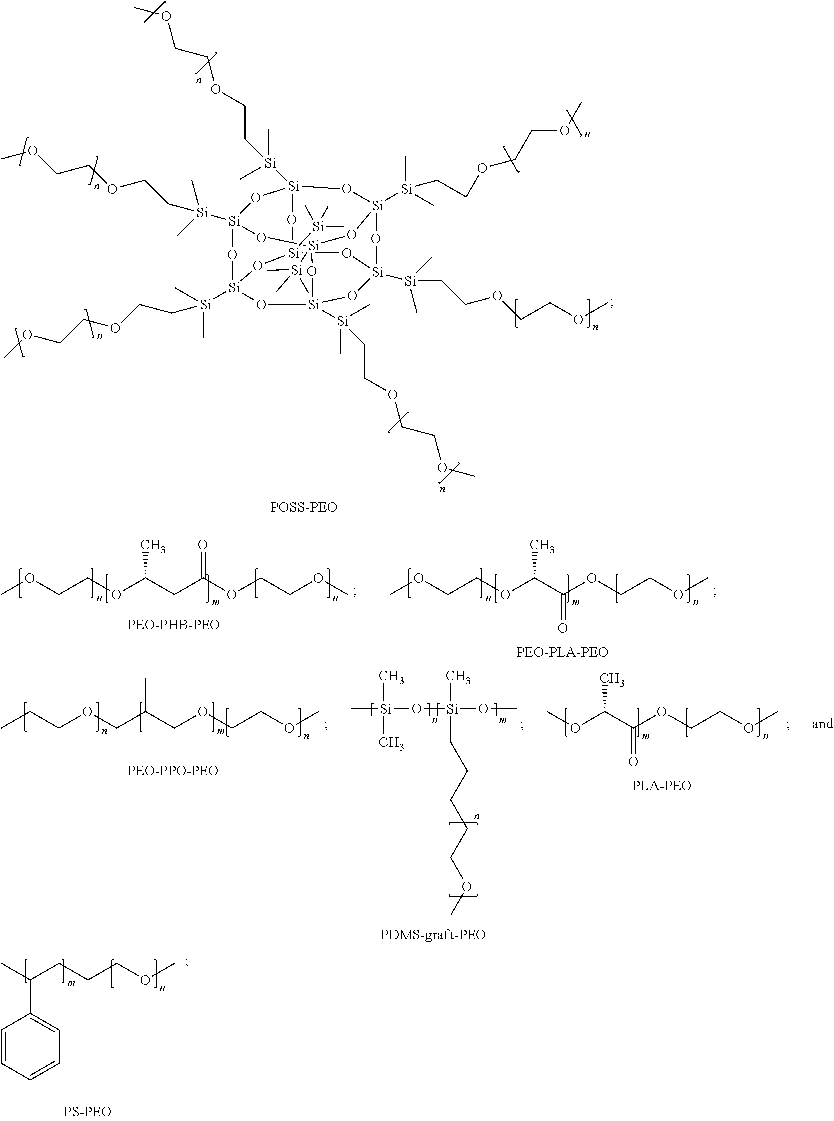

where each of “m” and “n”, where applicable, may have a value from 3 to 5000.

The hydrophilic component present in the corona of the micellar particle may aid in preventing or minimising nonspecific absorption of biomolecules onto the micellar particle. By limiting the absorption of biomolecules such as proteins, the micellar particle may avoid clearance by the reticuloendothelial system of a host. This may increase the blood circulation lifetime of the micellar particle when in vivo and increasing the applicability of the micellar particle in biological studies. The hydrophilic component may be the PEO block of the amphiphilic polymer as mentioned above that contributes to the antifouling effect of the micellar particle.

The inorganic compound making up the shell portion of the micellar particle may be a ceramic selected from the group consisting of hydroxyapatite (HA), zirconia (ZrO₂), silica (SiO₂), titanium oxide (TiO₂) and alumina (Al₂O₃). Due to the presence of a shell on the micellar particle, the shell may protect the fluorescent molecule in the core from being exposed to an external environment. This may aid in minimizing the escape or degradation of the fluorescent molecule present in the core. Hence, the fluorescent molecule may not be significantly affected by the external environment, such that the micellar particle can be used in a wide range of environments and applications. In addition, since the fluorescent molecule can be protected from exposure to (soluble) oxygen molecules present in the external environment, the photostability of the fluorescent molecule may be enhanced or improved (as compared to the same fluorescent molecule but freely present in an appropriate solvent). Accordingly, the photo lifetime of the fluorescent molecule may be longer when compared to the solution (non-form of the fluorescent molecule. The photo lifetime of the encapsulated fluorescent molecule may be more than that of the non-encapsulated fluorescent molecule by at least about 10%, at least about 20%, at least about 30%, at least about 40% or at least about 50%.

Further, the presence of the shell may substantially prevent the micelle from dissociation even though the polymer concentration of the amphiphilic polymer falls below the critical micelle concentration. Hence, the shell may substantially inhibit the dissociation of the polymeric micelles during dilution.

The fluorescent molecule may be used in biomedical applications such as in bioimaging or biodetecting. The fluorescent molecule may be an organic molecule. The fluorescent molecule may be selected from the group consisting of Coumarin545T, DCJTB, acridine orange, proflavine, N-(30sulfopropyl)acridinium, phenylalanine, tryptophan, tyrosine, anthracene, 9-cyanoanthracene, 9,10-Diphenylanthracene, naphthalene, 1-anilino-8-naphthalene sulfonate, 6-propionyl-2-(dimethylaminonaphthalene), perylene, phenanthrene, pyrene, puranine, p-quaterphenyl, rubrene, p-terphenyl, [60] fullerene, [70] fullerene, auramine O, malachite green, crystal violet, 1,3,5,7,8-pentamethylpyrromethene-difluoroborate, disodium-1,3,5,7,8-pentamethylpyrromethene-2,6-disulfonate-difluoroborate, 1,3,5,7,8-pentamethyl-2,6-diethylpyrromethene-difluoroborate, 8-Acetoxymethyl-2,6-diethyl-1,3,5,7-tetramethyl pyrromethene fluoroborate, 1,2,3,5,6,7-hexamethyl-8-cyanopyrromethene-difluoroborate, 2,3,5,6-1H,4H-Tetrahydro-8-methylquinolizino-[9,9a,1-gh]-coumarin, 7-amino-4-methylcoumarin, 7-amino-4-trifluoromethylcoumarin, 7-dimethylamino-4-trifluoromethylcoumarin, 7-diethylamino-4-trifluoromethylcoumarin, 2,3,5,6-1H,4H-Tetrahydro-8-trifluormethylquinolizino-[9,9a,1-gh]coumarin, 7-diethylaminocoumarin, 7-ethylamino-4-trifluormethylcoumarin, cryptocyanine, Cy3, Cy5, Cy7, 1,1′0diethyl-2,2′-dicarbocyanine iodide, HITCI, indocyanine green, IR 140, cresyl violet, nile blue, nile red, oxazeine 1, oxazine 170, oxazine 750, 2,5-Bis-(4-biphenylyl)-1,3,4-oxadiazole, 2,5-Bis-(4-biphenylyl)-oxazole, 2-(1-Naphthyl)-5-phenyloxazole, 2-(4-Biphenylyl)-6-phenylbenzoxazole, 1,4-Di[2-(5-phenyloxazolyl)]benzene, 2,5-Diphenyloxazole, thionine, methylene blue, eosin Y, erythrosine B, fluorescein disodium salt uranin, dichlorofluorescein disodium salt, tetrachlorotetraiodofluorescein, pyronine Y, pyronine B, rhodamine B, Rhodamine 6G, rhodamine 101, rhodamine 110, sulforhodamine, sulforhodamine 101, tetramethylrhodamine, quinine sulfate, DAPI, N-methylcarbazole, 4-dicyanomethylene-2-methyl-6-(p-dimethylaminostyryl)-4H-pyran, DCM-OH and N-acetyl-L-tryptophanamide. The fluorescent molecule may not be a fluorescent polymer.

The fluorescent molecule may have a high quantum yield of at least 0.1, at least 0.2, at least 0.3, at least 0.4, at least 0.5, at least 0.6, at least 0.7, at least 0.8, or at least 0.9. The fluorescent molecule may have a quantum yield in the range of about 0.1 to about 0.99.

The micellar particle having a fluorescent molecule in the core may be used as a fluorescent probe, including labels and tags. The micellar particles may be used for imaging, tracking or detecting one or more targets such as a molecule, a cell, or an organism. As each type of fluorescent molecule emits a specific colour at a specific wavelength, the fluorescent colour emitted by the micellar particle can be varied by choosing an appropriate fluorescent molecule for encapsulation by the micellar particle. Multi-color emission may also be obtained by encapsulating more than one fluorescent molecule in the core. The different types of fluorescent molecules may have different absorption spectra such that different colors of emission from the micellar particle may be obtained using different external excitation light sources. The emission light from one fluorescent molecule may be absorbed by another fluorescent molecule in the same particle and cause a further fluorescence emission at a different wavelength from the other fluorescent molecule. Since energy may be transferred between the different fluorescent molecules in the same particle, the fluorescent spectra of the micellar, particle may be tunable over a wide range, even when they are to be excited by the same light source. For instance, when the same micellar particle contains both blue and green fluorescent molecules and the absorption spectrum of the green fluorescent molecule has a substantial overlap with the emission spectrum of the blue fluorescent molecule, the emission from the blue fluorescent molecule can be absorbed by the green fluorescent molecule, to varying degrees from partial absorption to complete absorption, depending on the extent of overlap between the emission and absorption spectra and the relative amount of each type of fluorescent molecules in the particle. The color of overall emission from the micellar particle may depend on the degree of the intra-particle energy transfer, and may vary from pure blue, green, red, to white or another color. Different emission colors thus may be obtained using one external excitation light source, such as a single UV light source.

In addition to the fluorescent molecule within the core of the micellar particle, a therapeutic agent can also be encapsulated within the core. The therapeutic agent can be released from the micellar particle in vivo in a controlled manner.

The surface of the micellar particle or the corona of the micellar particle may be functionalised with a linker group. The surface of the micellar particle or the corona of the micellar particle may be chemically modified with a linker group such as a carboxylic acid group or an amine group for conjugation with a specific ligand to associate with a target receptor or substrate. Where the hydrophilic component of the amphiphilic polymer is present in the corona portion, the hydrophilic component may be functionally or chemically modified for conjugation to a desired ligand.

The ligand may be any detection molecule that can interact with a desired target molecule, cell or organism so as to result in association of the ligand (and therefore the attached particles) and the target. The ligand may specifically bind to a molecule, including a molecule expressed on a cell surface such as a cell surface marker or a cell receptor, including when internalized in a multicellular organism, for example by injection or ingestion. Such binding can in certain circumstances result in uptake of the ligand and conjugated micellar particle into a cell.

The ligand may be selected from a nucleotide, a nucleic acid molecule including single-stranded or double-stranded DNA or RNA, a peptide or a protein including a hormone, a peptide ligand, an antibody, a receptor, an antigen, an epitope or a nucleic acid binding protein, a small molecule including an enzyme substrate or an analogue thereof, avidin, streptavidin, biotin, an aptamer, a saccharide including a monosaccharide or a polysaccharide.

The target may be a receptor or a substrate present on a cell or an organism. Where the target is a receptor or substrate, the receptor or substrate binds to or specifically recognizes the ligand. The receptor or substrate may be a nucleic acid, an enzyme, an antibody, an antigen or a nucleic acid binding protein.

The attachment of a ligand to micellar particles may be non-covalent, for example electrostatic, or it may be a covalent attachment. The covalent attachment may be direct, meaning there is a covalent bond formed between the amphiphilic molecule of the particle and the ligand, or it may be indirect, for example by a bifunctional spacer or linker group or molecule that covalently bonds with both the amphiphilic molecule of the particle and the ligand.

The ligand may be covalently attached to the micellar particle. For example, one of the particle or the ligand may be prepared having a reactive functional group that reacts with a complementary functional group in the other of the particle or ligand under conditions that result in formation of a covalent bond between the two complementary functional groups. For example, biotin-avidin, antibody-antigen, carbodiimide coupling chemistry and the like. If a bi-functional cross-linker or spacer is used, the linker or spacer may be chosen or prepared to have one reactive functional group that reacts with a complementary functional group on the ligand, and a second reactive functional group that reacts with a complementary functional group on the particle. The linker or spacer may be first reacted with one of the ligand and particle under conditions to form a covalent bond between the linker or spacer and the ligand or particle. The product of the reaction may then be purified, or it may directly be added to the other of the ligand and particle and reacted under conditions that allow, for attachment of the ligand to the particle via the spacer or linker.

It will be appreciated that bi-functional spacers or linkers will be chosen depending on the functional groups present in the ligand and the particle, including amino groups, thiol groups, carboxyl groups, carbonyl groups, aldehyde groups. The reactive functional groups in the spacer or linker may be photoreactive or may be an NHS-ester, a maleimide group, a phenyl azide group, a hydrazide group or an isocyanate group. The spacer or linker should not react with the particle in such a manner to interfere with the fluorescent properties of the fluorescent molecule.

Due to, the presence of the ligand, the micellar particle may be used for visualizing cells, for tracking cells, or for detecting specific molecules in the cells or tissues.

Different ligands may be attached to the particles. When different types of particles with different fluorescence response characteristics are conjugated with different types of specific ligands, different types of targets may be tracked or detected simultaneously, such as by exciting the samples with light of different wavelengths, or detecting different emission wavelengths.

The size of the micellar particle may vary and can be adjusted as desired during formation of the micellar particle. The average size and size distribution of the micellar particles may be tailored by the choice of the amphiphilic polymer, shell precursor molecules, concentrations or ratio of the amphiphilic polymer or shell precursor molecules in their respective solvents, the nature of the solvent, the reaction temperature or agitation of the solution during reaction. The particle size of the micellar particle may be in the micron-sized or in the nano-sized. The particle size of the micellar particle may be in the nano-sized such that the micellar particle is termed as a nanoparticle, or a nanocapsule. The size of the micellar particle may be selected to avoid clearance by the reticuloendothelial system in a host. The size of the micellar particle may be less than 100 nm, less than 50 nm, less than 40 nm, less than 30 nm, or less than 20 nm. The average particle size of the micellar particles may be about 10 nm, about 11 nm, about 12 nm, about 13 nm, about 14 nm, about 15 nm, about 16 nm, about 17 nm, about 18 nm, about 19 nm, about 20 nm, about 21 nm, about 22 nm, about 23 nm, about 24 nm or about 25 nm. The thickness of the shell portion may be in the range of about 5% to about 95% of the particle diameter.

The micellar particles may be formed by dissolving precursors for the amphiphilic polymer and/or shell together with the fluorescent molecule in a non-aqueous solvent, such as an organic solvent, to form a solution and mixing the solution with an aqueous liquid such as water. Instead of using amphiphilic polymer precursor (or monomers), the amphiphilic polymer itself may be used in the first instance. The reactant solution may further comprise a swelling agent to enlarge the core portion of the micellar particle. The non-aqueous solvent may be removed from the mixture. The micellar particles may be formed, such as by self-aggregation of the amphiphilic polymer, in the mixture. In addition, the shell may be formed from the shell, precursor to at least partially encapsulate the core portion of the micellar particle.

Without being limited to any particular theory, it may be expected that when surrounded by water molecules, the hydrophilic components of the amphiphilic polymer may tend to move towards the surrounding water molecules and may even form hydrogen bonds with the water molecules. In the process, the hydrophilic components of the amphiphilic polymer tangle with one another, forming the corona portion of the micellar particle. In contrast, the hydrophobic components tend to move away from the water molecules but towards each other. In the process, the hydrophobic components of the amphiphilic polymer interact with one another, forming the core portion of the micellar particle. As a result, the fluorescent molecule is encapsulated by the amphiphilic polymer.

The precursors for the amphiphilic polymer and/or shell may be first prepared in any suitable manner. It is to be noted that a precursor for a polymer or a molecule may include one or more different molecules that will inter react to form the desired molecule, or it may be the same molecule that is to be prepared.

The precursor molecules may be dissolved in a solvent, thus forming a precursor solution containing the amphiphilic polymer and/or shell. The solvent used may be an organic solvent in which the selected molecules are soluble in. Exemplary organic solvents include tetrahydrofuran (THF), dioxane, chloroform, dichloromethane, etc. Other types of solvents in which the selected molecules are soluble may also be used. Suitable solvents in each particular application may vary depending on the particular molecules and precursors involved in the particular application. For example, where the fluorescent molecule is coumarin 545T, dioxane was used as the organic solvent instead of THF as using THF will result in precipitation with bad morphology of the micellar particles. The solvent may have a relatively high vapor pressure at room temperature, such as when compared to water, the benefit of which will become clear below. To facilitate dissolution and uniform distribution of the molecules, the solution may be agitated such as by sonication or stirring.

The precursor solution may be mixed with an aqueous liquid, such as purified water, to form a liquid mixture.

The aqueous liquid may also be an aqueous solution containing a solute such as a salt, a base, or an acid. The salt may have different ionic strength. The base or acid may be added to adjust the pH of the aqueous solution. The aqueous liquid may also contain an organic solvent such as alcohol. As will be understood by persons skilled in the art, the composition of the aqueous liquid may affect the properties of the particles formed. For instance, the aqueous liquid may be selected to improve control of the particle size or particle size distribution, or both. Depending on the application, additional additives may be added to the solution.

In a different embodiment, the aqueous liquid may be added before all of the other ingredients have been added. Further, the precursor (or monomers) for the amphiphilic polymer may be dissolved in the aqueous liquid, which is then mixed with the precursor shell solution. In another embodiment, the precursors (or monomers) for the amphiphilic polymer and the shell may be first dissolved in different solvents to form two separate solutions, which are then mixed with an aqueous liquid. It should be noted that changing the sequence of the steps described above or the manner in which the precursor solution is prepared may affect the eventual particle sizes or the size distribution of the resultant micellar particle.

The precursor solution may be added to the aqueous fluid. The precursor solution may be added slowly, such as drop-wise. This may aid in improving the homogeneity of the particles such as the uniform distribution of the particle size or the chemical and physical structures of the particles.

The shell precursor may be a hydroxyapatite precursor, a zirconia precursor, a silica precursor, a titanium oxide precursor and an alumina precursor. Hydroxyapatite precursors may include calcium hydroxide and phosphoric acid. The zirconia precursor may be an organozirconium compound. The silica precursor may comprise silicon alkoxide. The silicon alkoxide may be of the following formula Si(OR)ₙ, in which R is an C₁₋₆alkyl group and n is either 3 or 4. When n is 3, the silicon alkoxide is a trialkoxysilane and may be selected from the group consisting of trimethoxysilane, triethoxysilane, tripropoxysilane, tributoxysilane, tripentoxysilane and trihexoxysilane. When n is 4, the silicon alkoxide is a tetraalkoxysilane and may be selected from the group consisting of, tetramethoxysilane, tetraethoxysilane (or commonly known as tetraethyl orthosilicate, TEOS), tetrapropoxysilane, tetrabutoxysilane, tetrapentoxysilane and tetrahexoxysilane. The titanium oxide precursor may be a titanium hydroxide or a titanium peroxide. The alumina precursor may be an aluminum salt comprising one or more anions selected from alkoxides, aryl oxides, carboxylates, halides, sulphate, nitrate, oxylates, and acetoacetonates.

The swelling agent may be an alkylbenzene such as trimethylbenzene and Toluene. For example, when the fluorescent molecule is DCJTB, trimethylbenzene was used as the swelling agent in an organic solvent such as THF.

The aqueous liquid mixture may be stirred or otherwise agitated to facilitate sufficient mixing and uniform distribution of the reactants.

The concentrations of the reactants in the reaction solution may be in the range of 0.01˜0.09 mg/15˜125 mg/15˜130 μL/0.1˜1.8 μL/2˜20 g for fluorescent molecule/amphiphilic polymer/silica precursor/organic solvent/aqueous liquid. Where amphiphilic polymer precursors are used, the concentration of 15˜125 mg above then refers to the concentration of the amphiphilic polymer when formed in solution.

The organic solvent may be removed from the mixture after the mixture is sufficiently mixed. The solvent may be removed in any suitable manner. A simple technique is to remove the solvent by evaporation. The evaporation technique may be used when the solvent has a relatively high vapor pressure at the evaporation temperature, as compared to water. The high vapor pressure may be advantageous for at least two reasons. One is to limit evaporation of water from the mixture and the other is to evaporate the solvent at a sufficiently high rate, thus reducing, the time required to remove the solvent. For example, with many organic solvents such as THF, the evaporation may be carried out at a sufficient rate at room temperature. When desired and appropriate, the mixture may be heated to accelerate the evaporation process. It should be noted that the evaporation temperature should be low enough to limit evaporation of water. As the organic solvent is removed from the mixture, the shell precursor may form the shell at the interface between the core and the corona of the micellar particle. For example, where the shell, precursor is a silica precursor such as tetramethoxysilane (TMOS), the TMOS may completely hydrolyse and condense at the interface between the core and corona of the micelles to form the shell.

To speed up the evaporation process, the mixture may be continuously agitated or stirred during evaporation. Further, evaporation may also be accelerated by pumping evaporated solvent vapor away from the surface of the liquid mixture. In some embodiments, stirring may also improve the homogeneity of the particles formed. For example, in some embodiments, stirring may prevent the formation of large aggregates of particles or coagulation of particles. It has been found that the evaporation rate of the organic solvent may also affect the homogeneity of the particles. In some embodiments, a slower evaporation rate may improve the homogeneity of the particles. In one embodiment, the non-aqueous solvent may be completely evaporated over the period of one to two days.

The remaining liquid after evaporation may be subjected to further processing, such as filtration, dilution, drying, etc, as can be understood by one skilled in the art for removal.

The fluorescent micellar particles formed may be extracted from the final solution, such as by drying, and stored for future use. When the fluorescent micellar particles are to be used, they may be dissolved in an aqueous solution again since biomedical applications are carried out in an aqueous environment. While the dried particles may coagulate, once re-dissolved, the coagulated particles will disintegrate and again form relatively uniformly sized particles. In some applications, it is desirable that the fluorescent particles are of uniform sizes.

A solution containing particles that have different emission spectra can be obtained by dissolving different types of fluorescent micellar particles in the same aqueous fluid such as purified water, where each type of fluorescent micellar particles are prepared as mentioned above but having a distinct emission spectrum. Thus, the solution may have multi-color fluorescence emission properties, and may be used for multi-target probing.

In one example, the fluorescent molecule is coumarin545T or DCJTB, the amphiphilic polymer is Pluronic® F127 having repeating blocks of PEO and PPO and the shell precursor is a silica precursor such as tetramethoxysilane. The resultant micellar particle may have the fluorescent molecules in the PPO core with the PEO tails in the corona portion and silica making up the shell portion.

The exemplary processes described herein are easy to perform since the reactants can be mixed together at substantially the same time and hence, the process can be performed in one step. The exemplary processes do not require extensive heating, expensive equipment, or strict or difficult handling procedures.

### BRIEF DESCRIPTION OF DRAWINGS

The accompanying drawings illustrate a disclosed embodiment and serves to explain the principles of the disclosed, embodiment. It is to be understood, however, that the drawings are designed for purposes of illustration only, and not as a definition of the limits of the invention.

FIG. 1(a) is a transmission electron microscopy (TEM) image having a scale bar of 100 nm showing Coumarin545T@SiO₂ nanocapsules while FIG. 1(b) is a TEM image having a scale bar of 100 nm showing DCJTB@SiO₂ nanocapsules.

FIG. 2 is a graph showing the UV-Vis and photoluminescent spectra of Coumarin545T@SiO₂.

FIG. 3 is a graph showing the UV-Vis and photoluminescent spectra of DCJTB@SiO₂.

FIG. 4 is a graph comparing the time-resolved photoluminescence decay of Coumarin545T dissolved in THF with that of Coumarin545T encapsulated inside the core of the nanocapsules.

FIG. 5 is a graph comparing the time-resolved photoluminescence decay of DCJTB dissolved in THF with that of DCJTB encapsulated inside the core of the nanocapsules.

FIG. 6(a) is a graph showing the hydrodynamic sizes of Coumarin545T@SiO₂ in the stability study while FIG. 6(b) shows, the hydrodynamic sizes of Coumarin545T@F127.

FIG. 7 is a graph showing the hydrodynamic diameters of Coumarin545T@SiO₂ and DCJTB@SiO₂ dispersed in 1×PBS containing 10% FBS as a function of incubation time at 37° C.

FIG. 8 is a bar graph showing the viabilities of Hela cells cultured in the presence of Coumarin545T@SiO₂ or DCTJB@SiO₂.

FIG. 9a to FIG. 9d are confocal images obtained at different channels of Hela cells after being incubated with Coumarin545T@SiO₂. FIG. 9a was obtained under bright field; FIG. 9b was obtained under FITC channel; FIG. 9c was obtained under DAPI channel and FIG. 9d was obtained under FITC and DAPI channels (showing a merged image).

FIG. 10a to FIG. 10d are confocal images obtained at different channels of Hela cells after being incubated with DCJTB@SiO₂. FIG. 10a was obtained under bright field; FIG. 10b was obtained under Texas Red channel; FIG. 10c was obtained under DAPI channel and FIG. 10d was obtained under FITC and DAPI channels (showing a merged image).

### EXAMPLES

Non-limiting examples of the invention will be further described in greater detail by reference to specific Examples, which should not be construed as in any way limiting the scope of the invention.

### Example 1

### Preparation of Green Light Emitting Coumarin@SiO₂ Nanocapsule

75 mg of Pluronic® F127 (PEO₁₀₆PPO₆₅PEO₁₀₀) was first dissolved in 800 μL dioxane to form a clear solution. 50 μL (1 mg/mL in dioxane) of Coumarin 545T and 65 μL of tetramethoxysilane (TMOS) were then added. All chemicals were obtained from Sigma-Aldrich of Missouri of St. Louis of the United States of America, except for Coumarin 545T and DCJTB (used in Example 2) which was obtained from Tokyo Chemical. Industry of Japan. Coumarin 545T is an organic dye molecule having the chemical structure below.

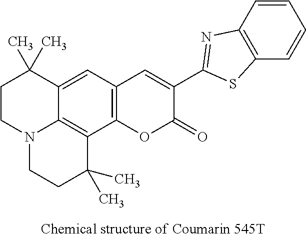 [SMILES: CC1(C)CCN2CCC(C)(C)c3c2c1cc1cc(-c2nc4ccccc4s2)c(=O)oc31]

The above mixture solution was injected into 10 mL of deionized water immersed in a water-bath under ultra sonication for 15 minutes. The solution was further sonicated for another 5 minutes, followed by stirring at room temperature for 4 days to evaporate off dioxane to ensure a complete hydrolysis of the TMOS at the interface between the core and corona of the F127 micelles. Fluorescent silica nanoparticles or nanocapsules consisting of a poly(propylene oxide) core, a silica shell and an exterior free poly(ethylene oxide) layer are formed. Due to the hydrophobicity of the Coumarin 545T molecules, the Coumarin 545T molecules are encapsulated inside the hollow core of the nanocapsules as discrete particles. Due to the presence of the exterior free poly(ethylene oxide) layer, the nanoparticles have excellent colloidal stability in an aqueous environment and are photostable.

### Example 2

### Preparation of Red Light Emitting Nanocapsule DCJTB@SiO₂Nanocapsule

75 mg of Pluronics® F127 was first dissolved into 600 μL tetrahydrofuran (THF) to form a clear solution. 50 μL (500 μg/mL in THF) of DCJTB, 20 μL of trimethylbenzene and 65 μL of TMOS were then added. Trimethylbenzene functions as a swelling agent to enlarge the hollow core of the nanocapsules to form the nanocapsule morphology of the micellar particle (DCJTB@SiO₂). DCJTB is an organic dye molecule having the chemical structure below.

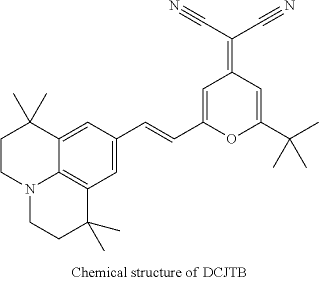 [SMILES: CC(C)(C)C1=CC(=C(C#N)C#N)C=C(/C=C/c2cc3c4c(c2)C(C)(C)CCN4CCC3(C)C)O1]

The above mixture solution was injected into 10 mL of deionized water immersed in a water-bath under ultra sonication for 15 minutes. The solution was further sonicated for another 5 minutes, followed by stirring at room temperature for 4 days to evaporate off THF to ensure a complete hydrolysis of TMOS at the interface between the core and corona of the F127 micelles. Similar to Example 1, fluorescent silica nanoparticles or nanocapsules consisting of a poly(propylene oxide) core, a silica shell and an exterior free poly(ethylene oxide) layer are formed. Due to the hydrophobicity of the Coumarin 545T molecules, the DCJBT molecules are encapsulated inside the hollow core of the nanocapsules as discrete particles.

### Preparation of Dicarboxylic Acid Modified F127

12.6 g of F127 was first dried in vacuum at 100° C. for 24 hours in order to remove adsorbed water. It was then dissolved in 60 mL of anhydrous dimethylacetamide (DMAC) while being stirred and heated to 70° C. Upon complete dissolution, 0.3 g of succinic anhydride was added into the solution and stirred rapidly at 70° C. under nitrogen atmosphere. The reaction mixture was then heated rapidly to 90° C. and stirred for 24 hours at this temperature. After cooling to room temperature, the final dark brown reaction mixture was precipitated against excess cold diethyl ether in a dropwise manner. The precipitates were dissolved in deionized water and dialyzed for 48 hours using dialysis tube with molecular weight cut-off (MWCO) of 1000 g/mol. The final solution was then freeze-dried to obtain dicarboxylic acid modified F127 block copolymer. The dicarboxylic acid modified F127 block copolymer may be used in place of the F127 of any of Example 1 or 2 above. As an Example, to introduce 20% carboxylic acid to the nanocapsule surface, 20% of F127 was replaced by dicarboxylic acid modified F127.

### Example 3

### Characterization of Silica Nanocapsules

The morphologies, optical properties, stabilities, antifouling properties, cytotoxicities and fluorescence images of the above silica nanocapsules formed from Examples 1 and 2 were characterized in this Example 3.

### Morphology of Nanocapsules

The morphologies of the Coumarin545T@SiO₂ and DCJTB@SiO₂ samples were determined using transmission electron microscopy (TEM) at a magnification of 36,000. FIG. 1(a) is a TEM image of Coumarin545T@SiO₂ nanocapsules while FIG. 1(b) is a TEM image of DCJTB@SiO₂ nanocapsules. As can be seen from FIG. 1(a) and FIG. 1(b), all of the nanocapsules have a core-shell structure with uniform size. The average core diameter of the samples was about 9 nm while the average outer diameter of the samples was about 15 nm.

### Optical Property Study

Quantum yield was measured with respect to the fluorescence standard solution, namely fluorescein (Ethanol; QY=0.79), and rhodamine B (Ethanol; QY=0.49). Fluorescein was used as a reference for Coumarin545T@SiO₂, whereas rhodamine B was used for DCJTB@SiO₂. The procedures involved diluting the fluorescent silica nanocapsules and the fluorescence standard solution with their respective solvent to have the same absorbance at excitation wavelength (460 nm for fluorescein; 490 nm for rhodamine B). Quantum yields for fluorescent silica nanocapsules were measured by dividing, the integrated emission area of their fluorescent spectrum against that of fluorecein or rhodamine B in Ethanol. As shown in Table 1, both of the nanoparticles exhibit high quantum yield of 0.99 for Coumarin545T@SiO₂ and 0.56 for DCJTB@SiO₂, suggesting that the fluorescent nanocapsules demonstrate the required high fluorescence brightness for fluorescence imaging applications. In addition, the lifetime of Coumarin545T@SiO₂ was determined to be 3.38 ns, while the lifetime of coumarin545T in THF was determined to be 2.86 ns. Similarly, the lifetime of DCJTB@SiO₂ was determined to be 3.40 ns, while the lifetime of DCJTB in THF was determined to be 2.50 ns.

TABLE 1
Optical properties of dye in THF and
encapsulated in silica nanocapsules

| | Coumarin 545T | | DCJTB | |
|---|---|---|---|---|
| | | nanocap- | | nanocap- |
| | THF | sules | THF | sules |
| λₑₘ (nm) | 504 | 520 | 605 | 610 |
| Fluorescence Lifetime (τ, ns) | 2.86 | 3.38 | 2.50 | 3.40 |
| Quantum Yield (QY) | 0.99 | 0.99 | 0.99 | 0.56 |
| fwhm (nm) | 35 | 70 | 73 | 90 |

The photoluminescence lifetimes of the Coumarin545T@SiO₂ and DCJTB@SiO₂ samples were measured using a Time Correlated Single Photon Counting (TCSPC) module (PicoQuant PicoHarp 300 of PicoQuant of Berlin, Germany). The Coumarin545T@SiO₂ and DCJTB@SiO₂ samples were excited by using 405 nm line of a picosecond pulsed laser diode (PicoQuant PDL 800-B). The photoluminescence was dispersed through a monochromator (Acton SpectroPro 2300i) and detected with a micro channel plate photomultiplier tube (MCP-PMT) detector (Hamamatsu R3809U-50).

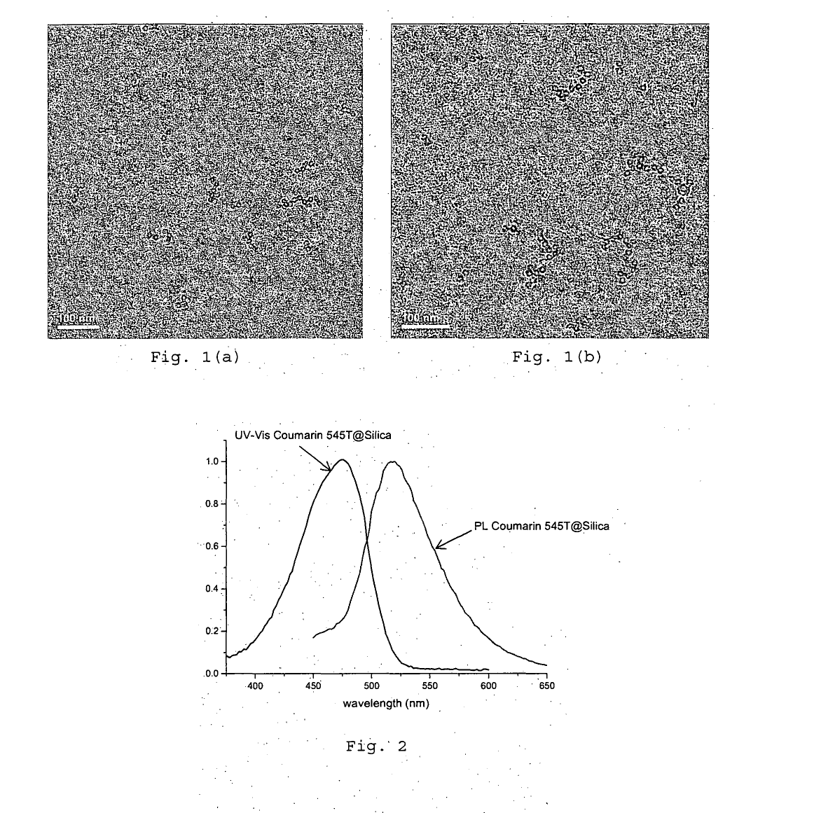

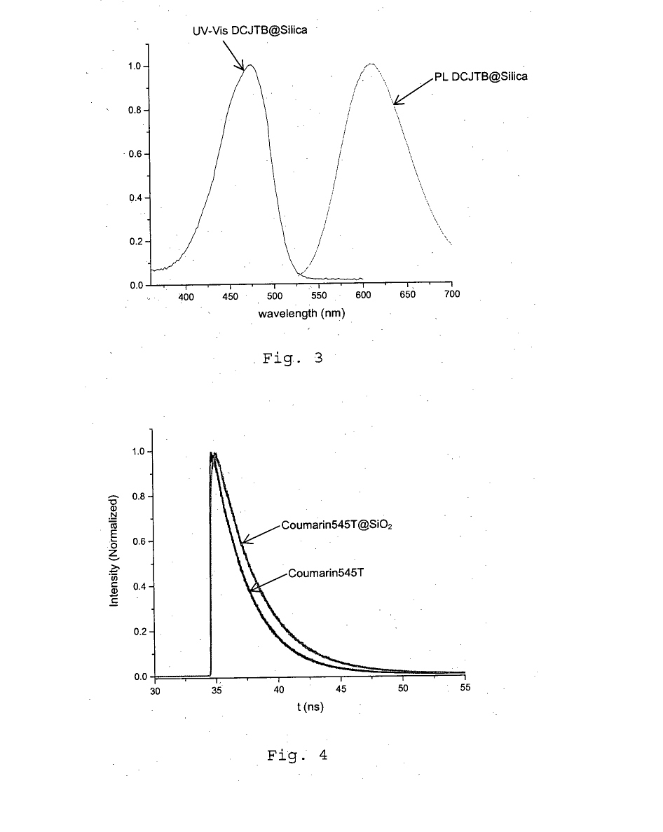

The UV-Vis and photoluminescent spectra of Coumarin545T@SiO₂ are shown in FIG. 2 while those of DCJTB@SiO₂ are shown in FIG. 3.

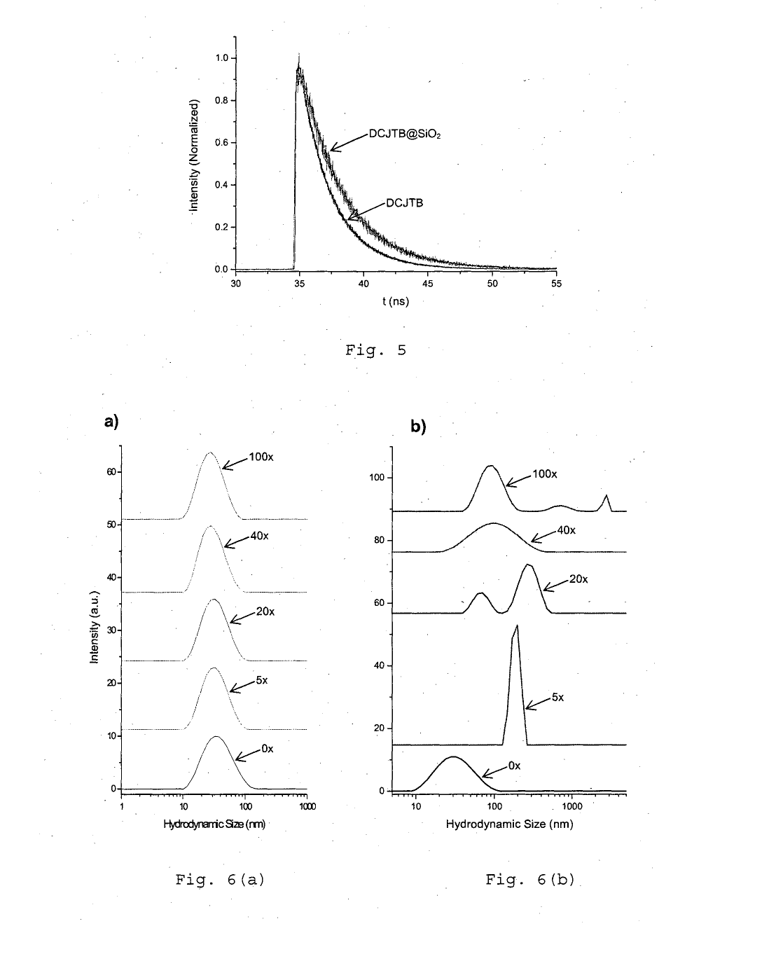

FIG. 4 is a graph showing the time-resolved photoluminescence decay of Coumarin545T dissolved in THF and that when encapsulated inside the core of the nanocapsules. FIG. 5 is a graph showing the time-resolved photoluminescence decay of DCJTB dissolved in THF and that when encapsulated inside the core of the nanocapsules. It was observed that all photoluminescence decay curves could be well fitted with a single exponential decay function and the obtained lifetimes of fluorescent nanocapsules are significantly larger than that of their corresponding dye solutions. Based on these figures, when compared to the photoluminescence behavior of the dye solutions (Coumarin545T or DCJTB) in their respective solvents, there is a significant red-shift in the emission peak for both of the silica nanocapsules, which should be ascribed to the π-π stacking of the dye molecules by encapsulation. The emission spectra of the nanocapsules are also broader than the dye solutions in their respective solvents. The silica coating could lead to improvement in the photostability of the dye molecules by limiting their contact with the outside environment, especially soluble oxygen molecules.

### Stability Study

The aqueous suspensions of the Coumarin545T@SiO₂ nanocapsules and Coumarin545T@F127 were diluted for 5 to 100 times against deionized water, and then stirred at room temperature for 24 hours before measurements of the hydrodynamic sizes by dynamic light scattering. The Coumarin545T@F127 sample was prepared in a similar way to Coumarin545T@SiO₂ nanocapsules but without the use of TMOS silica precursor. From FIG. 6(b), it can be observed that the hydrodynamic size of Coumarin545T@F127 significantly increased with a much more broader size distribution when diluted from 5 to 100 times, indicating the disintegration of the polymeric micelles. On the contrary, FIG. 6(a) showed that the hydrodynamic size of Coumarin545T@SiO₂ remained consistent with dilution from 5 to 100 times, indicating that the silica shell could successfully inhibit the dissociation of the polymeric micelles during dilution. Hence, unlike polymeric micelles which spontaneously dissociate when they are diluted below the critical micelle concentration (CMC), the silica shell of the fluorescent nanocapsules effectively prevented the micelles from dissociation even though the polymer concentration fell below the CMC.

### Antifouling Property Study

The antifouling behaviors of Coumarin545T@SiO₂ and DCJTB@SiO₂ nanocapsules were evaluated by monitoring their hydrodynamic size changes upon incubating the nanocapsules in 1×PBS with 10 vol % FBS at 37° C. The final concentration of the nanocapsules in the solution was 1 mg/mL. Dynamic Light Scattering (DLS) was used to monitor the hydrodynamic size changes during the incubation period.

As shown in FIG. 7, there was no significant variation in the hydrodynamic diameters of the Coumarin545T@SiO₂ and DCJTB@SiO₂ nanocapsules during the incubation period of 22 hour, indicating that the nanocapsules remained stable without aggregation in the presence of the serum proteins, demonstrating their antifouling behavior due to the presence of free hydrophilic poly(ethylene oxide) chains on the exterior surface that prevents nonspecific adsorption of biomolecules.

On the other hand, nanoparticles without suitable surface modification can easily aggregate into large particles and absorb protein on the surface, which unfortunately speeds up reticuloendothelial clearance and shortens blood circulation lifetime, limiting their use in biological studies. In addition, the particle size of the nanoparticles would increase significantly due to the nonspecific binding of proteins on silica surface.

### Cytotoxicity Study

The cytotoxicity of the fluorescent nanocapsules was studied by determining the viability of cervical cancer cells (Hela) using MTS assay. The Hela cells obtained from American Type Culture Collection (ATCC) were grown in culture medium at 37° C. in 5% CO₂. The culture medium contains completed DMEM with glucose (4500 mg/L), sodium pyruvate and L-glutamin, 10% FBS, 100 units Penicillin and 100 mg/ml of Streptomycin. The Hela cells were seeded into a 96 well plate at a density of 15,000 cells/well. The cells were then cultured at 37° C. in 5% CO₂ in the presence of growth medium containing fluorescent nanocapsules from 0 to 6 mg/mL. After incubation for 24 hours, the growth medium was discarded and replaced by MTS/phenazine methosulfate solution followed by incubation for another 1 hour. The optical absorbance of each well was then measured at 490 nm on a SpectraMax M2 Multi-Mode Microplate Reader (Molecular Devices). The results were normalized with respect to the result obtained without the addition of fluorescent nanocapsules. The data were averaged from three experiments for each fluorescent nanocapsules concentration.

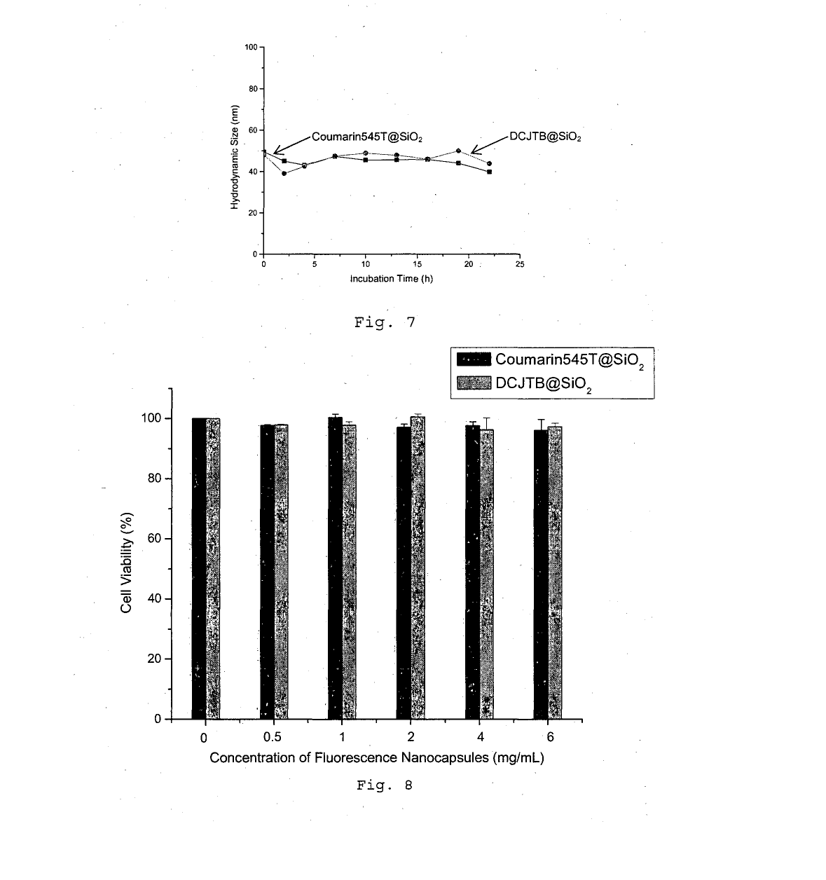

As shown in FIG. 8, the cell viability of Hela cells were close to 100% at the concentration of 6 mg/mL after incubation for 24 hours, suggesting that the nanocapsules are non-cytotoxic and safe for biomedical applications.

### Fluorescence Imaging Study

Hela cells were cultured in a 96-well plate with culture medium at 37° C. in 5% CO₂. The culture medium contains completed DMEM with 4500 mg/L of glucose, sodium pyruvate and L-glutamin, 10% FBS, 100 units of Penicillin and 100 mg/mL of Streptomycin. The Hela cells were seeded into chamber slides at a density of 15,000 cells/well. After 24 hours of incubation, the culture medium was replaced by fresh DMEM containing fluorescent nanocapsules at 2.4 mg/mL. After 1 hour of incubation in the presence of fluorescent nanocapsules, each well was washed with PBS for three times to remove supernatant nanoparticles. The cells were then mounted using culture medium with DAPI. The cells then were imaged by using an inverted microscope (Nikon, Eclipse Ti). The corresponding fluorescent image was acquired at FITC channel (ex 488 nm, em 525 nm) for coumarin545T@SiO₂ and Texas Red Channel (ex 595 nm, em 615 nm) for DCJTB@SiO₂.

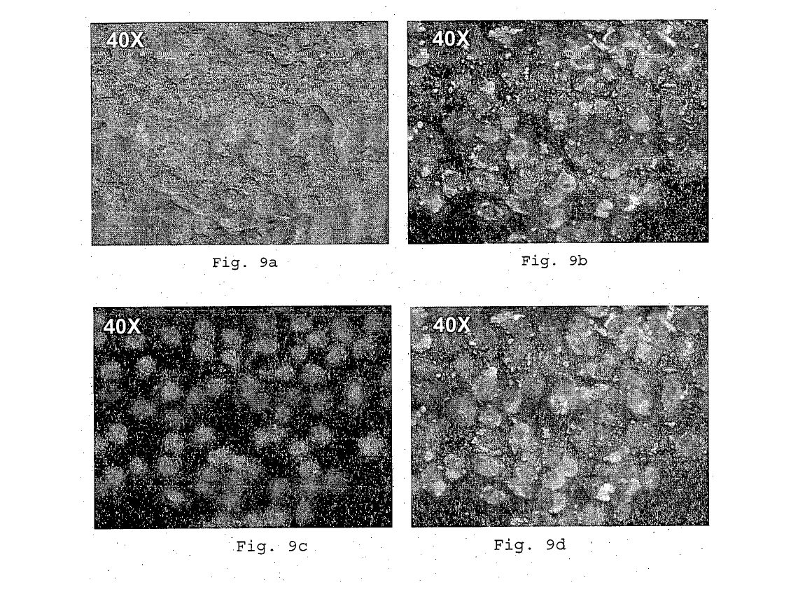

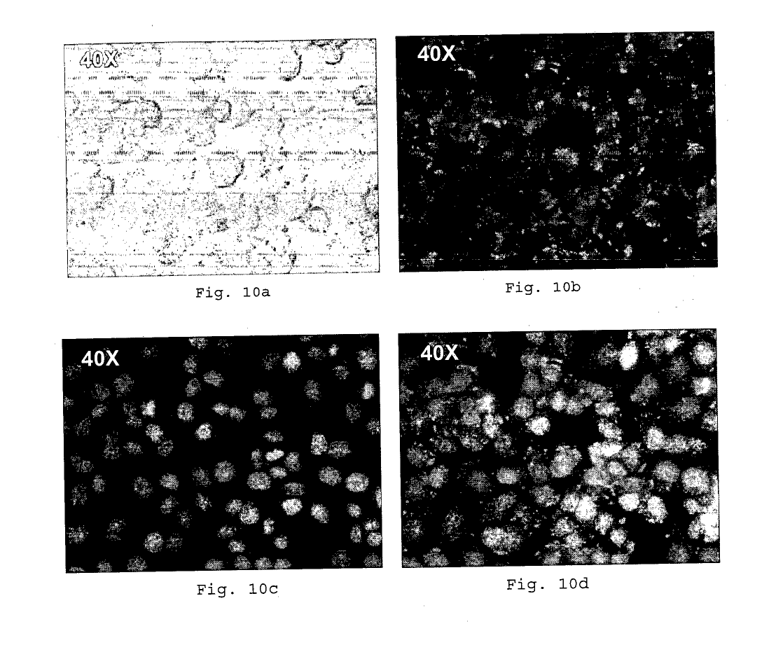

As can be seen in FIG. 9 and FIG. 10, the fluorescent images acquired at FITC channel (ex 488 nm, em 525 nm) for Coumarin545T@SiO₂ (FIG. 9) and Texas Red Channel (ex 595 nm, em 615 nm) for DCJTB@SiO₂ (FIG. 10) clearly demonstrated that both of the green and red nanoparticles can efficiently internalize in the cytoplasm region of the Hela cells, which showed great potential to be used as fluorescence cellular imaging probes.

### Comparative Example

A stock solution of poly(2-methoxy-5-(2-ethyl-hexyloxy)-1,4-phenylene vinylene) (MEH-PPV), poly(2-(2′-phenyl-4′,5′-di(3″-methyl-butoxy)-phenyl-1,4-phenylenevinylene)) (BP-PPV) and poly(9,9-dihexylfluorenyl-2,7-diyl) (C6PF) in THF were prepared by dissolving the respective polymers in THF to a concentration of 1.0 mg/ml. 75 μl of this stock solution was mixed with 75 mg of Pluronic® F127 (PEO₁₀₆PPO₇₀PEO₁₀₆) in 825 μl of THF to form a clear solution. 65 μl of TMOS was then added. The mixture solution was injected into a 10 g of deionized water immersed in water-bath under ultra sonication in three minutes. The solution was further sonicated for ten minutes, followed by stirring at room temperature for four days to evaporate off THF and ensure a complete hydrolysis of TMOS at the interface between the core and corona of the micelles.

The particles formed in this comparative example exhibited very low quantum yield of less than 10%, which significantly limits the use of these particles as effective emitters especially in experiments requiring high brightness of the fluorophores such as long-term cell tracing, animal study and clinical surgery. Hence, the applications of such fluorophores are severely limited due to the low quantum yields.

### Applications

The disclosed micellar particles may be used in biomedical applications such as bioimaging or biodetection.

The disclosed micellar particles may have low toxicity, good colloidal stability and may be capable of emitting distinctive fluorescence light upon excitation with high quantum yield and good photostability. Advantageously, the use of fluorescent molecules as disclosed herein may result in nanocapsules having a high quantum yield not seen in the prior art, which then increases the range of applications that can be employed with the disclosed nanocapsules.

The disclosed micellar particles may have a hydrophilic group on the corona portion of the particles to impart the desired colloidal stability, low cytotoxicity and enhanced blood circulation property. In addition, the disclosed micellar particles may be in the nano-size and may demonstrate good antifouling property, which could prevent the nanocapsules from non-specific binding to circulating proteins in a host and hence avoid clearance by the reticuloendothelial system of a host.

Fluorescent molecules that are encapsulated in the core of the micellar particles are protected from environmental degradation, leading to the micellar particles having good photostability.

The amphiphilic polymer making up the micellar polymer may be modified with a chemical group that can react with or bind to a ligand, which in turns can bind to a target receptor or substrate in vivo, allowing for bioimaging or biodetection.

The disclosed micellar particles may be made in a facile one-pot method whereby fluorescent silica nanocapsules encapsulating fluorescent molecules are formed.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A micellar particle having at least a core-shell configuration, wherein a fluorescent molecule is encapsulated within said core, said core being formed from a hydrophobic component of an amphiphilic polymer and wherein said shell is formed from an inorganic compound.

2. The micellar particle as claimed in claim 1, further comprising a corona that extends from said shell.

3. The micellar particle as claimed in claim 2, wherein a hydrophilic component of said amphiphilic polymer forms the corona of said micellar particle.

4. The micellar particle as claimed in claim 3, wherein said amphiphilic polymer is a block copolymer or a block terpolymer.

5. The micellar particle as claimed in claim 3, wherein said amphiphilic polymer comprises a polyethylene-oxide monomer.

6. The micellar particle as claimed in claim 5, wherein said polyethylene-oxide functionalized polymer is selected from the group consisting of polyhedral oligosilsesquioxanes-PEO (POSS-PEO), PEO-polyhydroxybutyrate-PEO (PEO-PHB-PEO), polylactic-acid-PEO (PLA-PEO), PEO-PLA-PEO, PEO-polypropyleneoxide-PEO (PEO-PPO-PEO), polydimethylsiloxane-graft-PEO (PDMS-graft-PEO) and polystyrene-PEO (PS-PEO).

7. The micellar particle as claimed in claim 1, wherein said inorganic compound is a ceramic selected from the group consisting of hydroxyapatite, zirconia, silica, titanium oxide and alumina.

8. The micellar particle as claimed in claim 1, wherein said fluorescent molecule is an organic molecule.

9. The micellar particle as claimed in claim 8, wherein said fluorescent molecule is selected from the group consisting of Coumarin545T, DCJTB, acridine orange, proflavine, N-(30sulfopropyl)acridinium, phenylalanine, tryptophan, tyrosine, anthracene, 9-cyanoanthracene, 9,10-Diphenylanthracene, naphthalene, 1-anilino-8-naphthalene sulfonate, 6-propionyl-2-(dimethylaminonaphthalene), perylene, phenanthrene, pyrene, puranine, p-quaterphenyl, rubrene, p-terphenyl, [60] fullerene, [70] fullerene, auramine O, malachite green, crystal violet, 1,3,5,7,8-pentamethylpyrromethene-difluoroborate, disodium-1,3,5,7,8-pentamethylpyrromethene-2,6-disulfonate-difluoroborate, 1,3,5,7,8-pentamethyl-2,6-diethyl pyrromethene-difluoroborate, 8-Acetoxymethyl-2,6-diethyl-1,3,5,7-tetramethyl pyrromethene fluoroborate, 1,2,3,5,6,7-hexamethyl-8-cyanopyrromethene-difluoroborate, 2,3,5,6-1H,4H-Tetrahydro-8-methylquinolizino-[9,9a,1-gh]-coumarin, 7-amino-4-methylcoumarin, 7-amino-4-trifluoromethylcoumarin, 7-dimethylamino-4-trifluoromethylcoumarin, 7-diethylamino-4-trifluoromethylcoumarin, 2,3,5,6-1H,4H-Tetrahydro-8-trifluormethylquinolizino-[9,9a,1-gh]coumarin, 7-diethylaminocoumarin, 7-ethylamino-4-trifluormethylcoumarin, cryptocyanine, Cy3, Cy5, Cy7, 1,1′0diethyl-2,2′-dicarbocyanine iodide, HITCI, indocyanine green, IR 140, cresyl violet, nile blue, nile red, oxazeine 1, oxazine 170, oxazine 750, 2,5-Bis-(4-biphenylyl)-1,3,4-oxadiazole, 2,5-Bis-(4-biphenylyl)-oxazole, 2-(1-Naphthyl)-5-phenyloxazole, 2-(4-Biphenylyl)-6-phenylbenzoxazole, 1,4-Di[2-(5-phenyloxazolyl)]benzene, 2,5-Diphenyloxazole, thionine, methylene blue, eosin Y, erythrosine B, fluorescein disodium salt uranin, dichlorofluorescein disodium salt, tetrachlorotetraiodofluorescein, pyronine Y, pyronine B, rhodamine B, Rhodamine 6G, rhodamine 101, rhodamine 110, sulforhodamine, sulforhodamine 101, tetramethylrhodamine, quinine sulfate, DAPI, N-methylcarbazole, 4-dicyanomethylene-2-methyl-6-(p-dimethylaminostyryl)-4H-pyran, DCM-OH and N-acetyl-L-tryptophanamide.

10. The micellar particle as claimed in claim 2, wherein said corona of said micellar particle is functionalized with a linker group.

11. The micellar particle as claimed in claim 10, wherein said linker group is selected from a carboxylic acid group or an amine group.

12. The micellar particle as claimed in claim 1, wherein said micellar particle has a particle size in the nano-sized range.

13. The micellar particle as claimed in claim 12, wherein the particle size of said micellar particle is less than 10 nm.

14. The micellar particle as claimed in claim 1, wherein said shell has a thickness that is in the range of 5% to 95% of the diameter of said micellar particle.

15. The micellar particle as claimed in claim 1, further comprising a therapeutic agent encapsulated within said core.

16. A process for forming a micellar particle having at least a core-shell configuration comprising:
a. mixing a reactant solution comprising an amphiphilic polymer or monomers thereof, a fluorescent molecule and shell precursors in an organic solvent; and
b. mixing said reactant solution from operation (a) with an aqueous liquid to form said micellar particle in which said fluorescent molecule is encapsulated within said core, said core being formed from a hydrophobic component of said amphiphilic polymer and wherein said shell is formed from said shell precursors.

17. The process as claimed in claim 16, wherein said organic solvent is selected from the group consisting of tetrahydrofuran, dioxane, chloroform and dichloromethane.

18. The process as claimed in claim 16, wherein said shell precursor is selected from the group consisting of a hydroxyapatite precursor, a zirconia precursor, a silica precursor, a titanium oxide precursor and an alumina precursor.

19. The process as claimed in claim 16, wherein said reactant solution further comprises a swelling agent.

20. Use of a micellar particle having at least a core-shell configuration, wherein a fluorescent molecule is encapsulated within said core, said core being formed from a hydrophobic component of an amphiphilic polymer and wherein said shell is formed from an inorganic compound, as a bioimaging agent or a biodetection agent.
